(19) United States
(12) Patent Application Publication
(10) Pub. No.: US 2018/0061098 A1
(43) Pub. Date: Mar. 1, 2018

(54) ELECTRONIC DEVICE AND METHOD FOR PROVIDING USER'S ACTIVITY INFORMATION THEREOF

(71) Applicant: Samsung Electronics Co., Ltd., Gyeonggi-do (KR)

(72) Inventors: Hyo-Gil KIM, Gyeonggi-do (KR); Yonghak Lee, Gyeonggi-do (KR); Keun-Sang Han, Gyeonggi-do (KR); Bumjoon Kim, Seoul (KR); Han-Jib Kim, Gyeonggi-do (KR)

(21) Appl. No.: 15/684,602

(22) Filed: Aug. 23, 2017

(30) Foreign Application Priority Data
Aug. 30, 2016 (KR) 10-2016-0110767

(51) Int. Cl. G06T 11/20; A63B 71/06; A63B 24/00; A61B 5/0205; A61B 5/00

(52) U.S. Cl. CPC G06T 11/206; A63B 71/0622; A63B 24/0021; A63B 24/0062; A61B 5/02055; A61B 5/681; A61B 5/14532; A63B 2220/12; A63B 2220/56; A63B 2230/06; A63B 2220/40; A63B 2220/89; A63B 2220/836; A63B 2071/0691

(57) ABSTRACT

An electronic device and a method for providing user activity information are provided. The electronic device includes at least one sensor, a memory, a display, and at least one processor functionally coupled with the at least one sensor, the memory, and the display. The at least one processor is configured to control the at least one sensor to collect activity information about a user's activity, control the memory to store the collected activity information, control the display to display activity information collected on a particular date, using a 24-hour graph in response to an activity information display request, and control the display to display, when the activity information continues from a previous day of the particular date to the particular date and is of a particular type, previous-day activity information of the particular type on the 24-hour graph.

### PRIORITY

This application claims priority under 35 U.S.C. §119(a) to Korean Patent Application Serial No. 10-2016-0110767, which was filed in the Korean Intellectual Property Office on Aug. 30, 2016, the entire disclosure of which is incorporated herein by reference.

### BACKGROUND

### 1. Field of the Disclosure

The present disclosure relates to an electronic device and method for providing user activity information by recording and/or analyzing the user activity information.

### 2. Description of the Related Art

Electronic devices (e.g., mobile terminals, smart phones, wearable devices, etc.) may provide various functions. A smart phone may provide, in addition to a basic voice communication function, an Internet access function, a music or video play function, a camera function, a navigation function, and a messenger function. Recent electronic devices may track a user's activity, store (or record) a user's activity information, and visually provide the stored user activity information to the user. Wearable electronic devices, which are worn on a user's body part (e.g., a wrist), may measure the user's activity (e.g., walking, running, sleeping, heart rate, stair climbing, cycling, climbing, etc.), and provide a measurement result and/or an analysis result (e.g., sleeping hours, exercise hours, and calories burned).

Electronic devices may store the user's activity information based on universal time coordinated (UTC). As such, when the user's activity information is stored based on the UTC, the electronic devices may continuously record the user's activity information though time zone changes but cannot display the user's activity information in accordance with time information of a region (hereafter, referred to as a local time) where a user is located.

The electronic devices may provide the user's activity information based on 24 hours. The electronic devices may provide the user's activity information using a graph (hereafter, referred to as an activity information graph). The activity information graph may use a circular, oval or quadrangular graph.

However, when displaying the user's activity information of 24 hours on a single activity information graph, the electronic devices cannot display on the graph, activity information which starts before the 24 hour period and ends after the 24 hour period. When the user goes to bed at 10 PM on August 24 and gets up at 7 AM on August 25, the activity information graph is initialized based on 24:00. Accordingly, at 24:00 on August 24 (=00:00 on August 25), sleep information from 10 PM on August 24 to 24:00 on August 24 is not displayed on the activity information graph. In this case, to check the sleep start time, the user needs to switch to yesterday's activity information graph.

### SUMMARY

According to an aspect of the present disclosure, when activity information of a particular type is continuous from the day before, an electronic device displays yesterday's activity information on today's graph.

According to another aspect of the present disclosure, when yesterday's activity information of a particular type and today's activity information overlap, an electronic device displays today's activity information on the top of a display, and when the activity information of the particular type is selected, the electronic device displays the activity information of the particular type on the top of a display.

According to another aspect of the present disclosure, an electronic device provides a user's activity information based on local time.

In accordance with an aspect of the present disclosure, an electronic device is provided which includes at least one sensor, a memory, a display, and at least one processor functionally coupled with the at least one sensor, the memory, and the display. The at least one processor is configured to control the at least one sensor to collect activity information about a user's activity, control the memory to store the collected at least one activity information, control the display to display activity information collected on a particular date, using a 24-hour graph in response to an activity information display request, and control the display to display, when the activity information continues from a previous day of the particular date to the particular date and is of a particular type, previous-day activity information of the particular type on the 24-hour graph.

In accordance with another aspect of the present disclosure, a method is provided for providing activity information in an electronic device. The method includes collecting and storing activity information of a user's activity, and displaying at least one activity information collected on a particular date, using a 24-hour graph in response to an activity information display request. Displaying the activity information using the graph include when activity information continues from a previous day of the particular date to the particular date and is of a particular type, displaying previous-day activity information of the particular type on the 24-hour graph.

In accordance with another aspect of the present disclosure, a computer-readable recording medium is provided which stores at least one instruction, the at least one instruction, when executed by at least one processor, causes the at least one processor to collect and store activity information of a user's activity, and display at least one activity information collected on a particular date, using a 24-hour graph in response to an activity information display request, and when activity information of a particular type continues from a previous day of the particular date to the particular date, display previous-day activity information on the 24-hour graph.

### BRIEF DESCRIPTION OF THE DRAWINGS

The above and other aspects, features, and advantages of the present disclosure will be more apparent from the following description taken in conjunction with the accompanying drawings, in which:

FIG. 1 is a block diagram of an electronic device in a system, according to an embodiment of the present disclosure;

FIG. 2 is a block diagram of an electronic device, according to an embodiment of the present disclosure;

FIG. 3 is a block diagram of a program module, according to an embodiment of the present disclosure;

FIG. 4 is a block diagram of an electronic device, according to an embodiment of the present disclosure;

FIG. 5 is a flowchart of a method for storing and providing user activity information in an electronic device, according to an embodiment of the present disclosure;

FIG. 6 is a diagram of activity information displayed when moving into a region where a time offset increases, according to an embodiment of the present disclosure;

FIG. 7 is a diagram of activity information displayed when moving into a region where a time offset decreases, according to an embodiment of the present disclosure;

FIG. 8A is a flowchart of a method for providing user activity information in an electronic device, according to an embodiment of the present disclosure;

FIG. 8B is a flowchart of a method for displaying user activity information in an electronic device using a circular graph, according to an embodiment of the present disclosure;

FIGS. 8C and 8D are diagrams of a circular graph displaying user activity information of an electronic device, according to an embodiment of the present disclosure;

FIG. 9 is a diagram showing the display of user activity information on an electronic device, according to an embodiment of the present disclosure; and

FIG. 10 is a diagram showing the display of user activity information on an electronic device, according to another embodiment of the present disclosure.

### DETAILED DESCRIPTION

Certain embodiments of the present disclosure are described in greater detail with reference to the accompanying drawings. The same or similar components may be designated by the same or similar reference numerals although they are illustrated in different drawings. Detailed descriptions of constructions or processes known in the art may be omitted to avoid obscuring the subject matter of the present disclosure.

The terms used herein are defined in consideration of functions of the present disclosure and may vary depending on a user's or an operator's intention and usage. Therefore, the terms used herein should be understood based on the descriptions made herein. It is to be understood that the singular forms “a,” “an,” and “the” also include plural referents unless the context clearly dictates otherwise. In the present disclosure, an expression such as “A or B,” “at least one of A and B” or “one or more of A and B” may include all possible combinations of the listed items. Expressions such as “first,” “second,” “primarily,” or “secondary,” as used herein, may represent various elements regardless of order and/or importance, and do not limit the corresponding elements. The expressions may be used for distinguishing one element from another element. When it is described that an element (such as a first element) is operatively or communicatively “coupled to” or “connected to” another element (such as a second element), the element may be directly connected to the other element or may be connected through another element (such as a third element).

The expression “configured (or set) to”, as used in the present disclosure, may be used interchangeably with, for example, “suitable for,” “having the capacity to,” “designed to,” “adapted to,” “made to,” or “capable of”, according to the situation. The term “configured (or set) to” does not only refer to “specifically designed to” in hardware. Alternatively, in some situations, the expression “apparatus configured to” may refer to a situation in which the apparatus “may” operate together with another apparatus or component. The phrase “a processor configured (or set) to perform A, B, and C” may be a dedicated processor, a general-purpose processor (such as a central processing (CPU) or an application processor (AP)) that may perform a corresponding operation by executing at least one software program stored in a dedicated processor (such as an embedded processor) for performing a corresponding operation or in a memory device.

An electronic device, according to an embodiment of the present disclosure, may be for example, at least one of a smart phone, a tablet PC, a mobile phone, a video phone, an e-book reader, a desktop PC, a laptop PC, a notebook computer, a workstation, a server, a PDA, a portable multimedia player (PMP), an MPEG 3 (MP3) player, medical equipment, a camera, and a wearable device, and the like, but is not limited thereto. The wearable device may include at least one of an accessory type (e.g., a watch, a ring, a bracelet, an ankle bracelet, a necklace, eyeglasses, a contact lens, or a head-mounted-device (HMD)), a fabric or clothing embedded type (e.g., electronic garments), a body attachable type (e.g., a skin pad or a tattoo), and an implantable circuit, and the like, but is not limited thereto. The electronic device may be at least one of, for example, a television, a digital versatile disc (DVD) player, an audio device, a refrigerator, an air-conditioner, a cleaner, an oven, a microwave oven, a washing machine, an air cleaner, a set-top box, a home automation control panel, a security control panel, a media box, an electronic dictionary, an electronic key, a camcorder, and an electronic frame, and the like, but is not limited thereto.

In an embodiment of the present disclosure, the electronic device may be at least one of various medical devices (such as, various portable medical measuring devices (a blood sugar level measuring device, a heartbeat measuring device, a blood pressure measuring device, or a body temperature measuring device), a magnetic resonance angiography (MRA) device, a magnetic resonance imaging (MRI) device, a computed tomography (CT) device, a scanning machine, and an ultrasonic wave device), a navigation device, a global navigation satellite system (GNSS), an event data recorder (EDR), a flight data recorder (FDR), a vehicle infotainment device, electronic equipment for a ship (such as, a navigation device for a ship and a gyro compass), avionics, a security device, a head unit for a vehicle, an industrial or home robot, a drone, an automated teller machine (ATM), a point of sales (POS) device, and an Internet of things (IoT) device (e.g., a light bulb, various sensors, a sprinkler device, a fire alarm, a thermostat, a street light, a toaster, sports equipment, a hot water tank, a heater, and a boiler), and the like, but is not limited thereto.

According to an embodiment of the present disclosure, the electronic device may be at least one of a portion of furniture, building/construction or vehicle, an electronic board, an electronic signature receiving device, a projector, and various measuring devices (e.g., water supply, electricity, gas, or electric wave measuring device), and the like, but is not limited thereto. An electronic device may be a flexible electronic device or a combination of two or more of the foregoing various devices. An electronic device is not limited to the foregoing devices, and may be embodied as a newly developed electronic device. The term “user”, as used herein, may refer to a person using an electronic device or a device using an electronic device (e.g., an artificial intelligence electronic device).

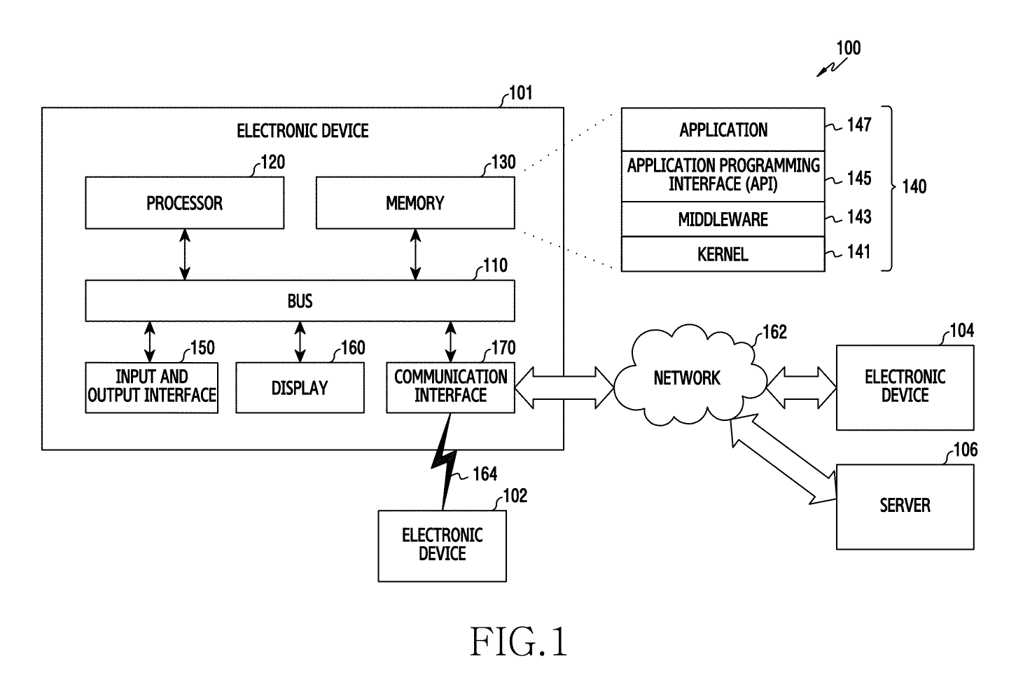

FIG. 1 is a block diagram of an electronic device in a system (100), according to an embodiment of the present disclosure.

Referring initially to FIG. 1, electronic devices 101, 102, 104 and/or a server 106 may be connected to each other via a network 162 and/or a wireless (e.g., short-range) communication 164. The electronic device 101 includes a bus 110, a processor (e.g., including processing circuitry) 120, a memory 130, an input/output interface (e.g., including input/output circuitry) 150, a display 160, and a communication interface (e.g., including communication circuitry) 170. The electronic device 101 may be provided without at least one of the components, or may include at least one additional component.

The bus 110 may include a circuit for connecting the components 120 through 170 and delivering communication signals (e.g., control messages or data) therebetween.

The processor 120 may include various processing circuitry, such as, for example, and without limitation one or more of a dedicated processor, a CPU, an AP, and a communication processor (CP). The processor 120, for example, may perform an operation or data processing with respect to control and/or communication of at least another component of the electronic device 101.

The memory 130 may include a volatile and/or nonvolatile memory. The memory 130, for example, may store commands or data relating to at least another component of the electronic device 101. According to an embodiment of the present disclosure, the memory 130 may store software and/or a program 140. The program 140 includes, for example, a kernel 141, middleware 143, an application programming interface (API) 145, and/or an application program (or applications) 147. At least part of the kernel 141, the middleware 143, or the API 145 may be referred to as an operating system (OS). The kernel 141 may control or manage system resources (e.g., the bus 110, the processor 120, or the memory 130) used for performing operations or functions implemented by the other programs (e.g., the middleware 143, the API 145, or the applications 147). Additionally, the kernel 141 may provide an interface for controlling or managing system resources by accessing an individual component of the electronic device 101 from the middleware 143, the API 145, or the applications 147.

The middleware 143, for example, may serve an intermediary role for exchanging data between the API 145 or the applications 147 and the kernel 141 through communication. Additionally, the middleware 143 may process one or more job requests received from the applications 147, based on their priority. The middleware 143 may assign a priority for using a system resource (e.g., the bus 110, the processor 120, or the memory 130) of the electronic device 101 to at least one of the applications 147, and process the one or more job requests. The API 145, as an interface through which the applications 147 controls a function provided from the kernel 141 or the middleware 143, may include, for example, at least one interface or function (e.g., an instruction) for file control, window control, image processing, or character control. The input/output interface 150, for example, may deliver commands or data input from a user or another external device to other component(s) of the electronic device 101, or output commands or data input from the other component(s) of the electronic device 101 to the user or another external device.

The display 160, for example, may include a liquid crystal display (LCD), a light emitting diode (LED) display, an organic light emitting diode (OLED) display, a microelectromechanical systems (MEMS) display, or an electronic paper display, and the like, but is not limited thereto. The display 160, for example, may display various content (e.g., texts, images, videos, icons, and/or symbols) to the user. The display 160 may include a touch screen, for example, and receive touch, gesture, proximity, or hovering inputs by using an electronic pen or a user's body part.

The communication interface 170 may include various communication circuitry and, for example, may set communications between the electronic device 101 and an external device (e.g., a first external electronic device 102, a second external electronic device 104, or a server 106. The communication interface 170 may communicate with the second external electronic device 104 or the server 106 over a network 162 through wireless communication or wired communication. The communication interface 170 may additionally communicate with the first external electronic device 102 using a short-range wireless communication connection 164.

The wireless communication, for example, may include cellular communication using at least one of long term evolution (LTE), LTE-advanced (LTE-A), code division multiple access (CDMA), wideband CDMA (WCDMA), universal mobile telecommunications system (UMTS), wireless broadband (WiBro), or global system for mobile communications (GSM). The wireless communication may include, for example, at least one of wireless fidelity (Wi-Fi), light fidelity (Li-Fi), Bluetooth™, Bluetooth™ low energy (BLE), Zigbee, near field communication (NFC), magnetic secure transmission, radio frequency (RF), and body area network (BAN). The wireless communication may include GNSS. The GNSS may include, for example, global positioning system (GPS), global navigation satellite system (GLONASS), Beidou navigation satellite system (Beidou), or Galileo (the European global satellite-based navigation system). Hereafter, the term GPS may be interchangeably used with the term GNSS. The wired communication, for example, may include at least one of universal serial bus (USB), high definition multimedia interface (HDMI), recommended standard 232 (RS-232), power line communications, and plain old telephone service (POTS). The network 162 may include a telecommunications network, for example, at least one of a computer network (e.g., LAN or WAN), the Internet, and a telephone network.

Each of the first and second external electronic devices 102 and 104 may be of the same type or of a different type from that of the electronic device 101. According to an embodiment of the present disclosure, all or part of operations executed in the electronic device 101 may be executed by another electronic device or a plurality of electronic devices (e.g., the electronic device 102 or 104, or the server 106). To perform a function or service automatically or by request, instead of performing the function or the service by the electronic device 101, the electronic device 101 may request at least part of a function relating thereto from the electronic device 102 or 104, or the server 106. The electronic device 102 or 104, or the server 106 may perform the requested function or an additional function and send its result to the electronic device 101. The electronic device 101 may provide the requested function or service by processing the received result. In doing so, for example, cloud computing, distributed computing, or client-server computing techniques may be used.

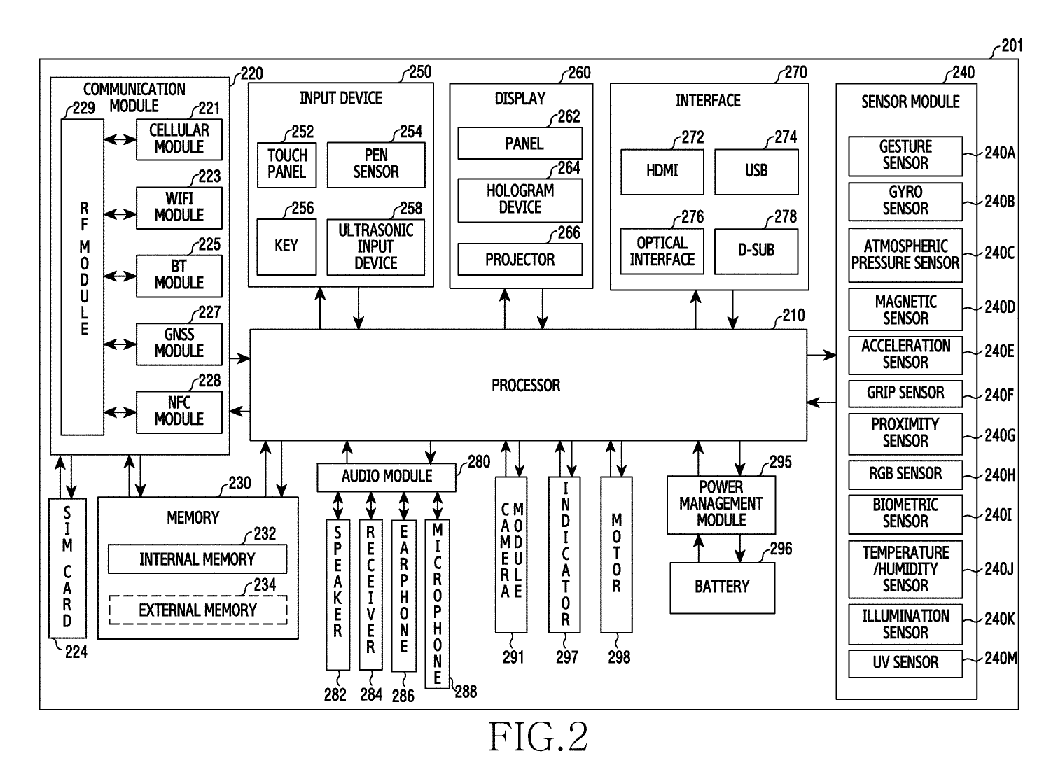

FIG. 2 is a block diagram of an electronic device, according to an embodiment of the present disclosure.

The electronic device 201, for example, may include all or part of the above-described electronic device 101 of FIG. 1. The electronic device 201 includes one or more processors (e.g., an AP) 210, a communication module 220, a subscriber identification module (SIM) 224, a memory 230, a sensor module 240, an input device 250, a display 260, an interface 270, an audio module 280, a camera module 291, a power management module 295, a battery 296, an indicator 297, and a motor 298.

The processor 210, for example, may include various processing circuitry and may control a plurality of hardware or software components connected to the processor 210, and also may perform various data processing and operations by executing an OS or an application program. The processor 210 may be implemented with a system on chip (SoC), for example. The processor 210 may further include a graphic processing unit (GPU) and/or an image signal processor. The processor 210 may include at least part (e.g., a cellular module 221) of the components illustrated in FIG. 2. The processor 210 may load commands or data received from at least one other component (e.g., a nonvolatile memory) into a volatile memory, process the commands, and store various data in the nonvolatile memory.

The communication module 220 may have the same or similar configuration as the communication interface 170 of FIG. 1. The communication module 220 may include various communication circuitry, such as, the cellular module 221, a Wi-Fi module 223, a Bluetooth™ (BT) module 225, a GNSS module 227, an NFC module 228, and an RF module 229. The cellular module 221, for example, may provide voice call, video call, short message service (SMS), or Internet service through a communication network. The cellular module 221 may identify and authenticate the electronic device 201 in a communication network by using the SIM 224. The cellular module 221 may perform at least part of a function that the processor 210 provides. The cellular module 221 may further include a CP. At least some (e.g., two or more) of the cellular module 221, the Wi-Fi module 223, the BT module 225, the GNSS module 227, and the NFC module 228 may be included in one integrated circuit (IC) or an IC package. The RF module 229, for example, may transmit/receive a communication signal (e.g., an RF signal). The RF module 229, for example, may include a transceiver, a power amp module (PAM), a frequency filter, a low noise amplifier (LNA), or an antenna. At least one of the cellular module 221, the Wi-Fi module 223, the BT module 225, the GNSS module 227, and the NFC module 228 may transmit/receive an RF signal through an additional RF module.

The SIM 224, for example, may include a card or an embedded SIM, and also may contain unique identification information (e.g., an integrated circuit card identifier (ICCID)) or subscriber information (e.g., an international mobile subscriber identity (IMSI)).

The memory 230 (e.g., the memory 130) may include at least one of an internal memory 232 or an external memory 234. The internal memory 232 may include at least one of, for example, a volatile memory (e.g., dynamic RAM (DRAM), static RAM (SRAM), or synchronous dynamic RAM (SDRAM)), and a non-volatile memory (e.g., one time programmable ROM (OTPROM), programmable ROM (PROM), erasable and programmable ROM (EPROM), electrically erasable and programmable ROM (EEPROM), mask ROM, flash ROM, flash memory, hard drive, and solid state drive (SSD)). The external memory 234 may include flash drive, for example, compact flash (CF), secure digital (SD), micro SD, mini SD, extreme digital (xD), multi-media card (MMC), or memory stick. The external memory 234 may be functionally or physically connected to the electronic device 201 through various interfaces.

The sensor module 240 may, for example, measure physical quantities or detect an operating state of the electronic device 201, and convert the measured or detected information into electrical signals. The sensor module 240 includes at least one of a gesture sensor 240A, a gyro sensor 240B, an atmospheric pressure sensor 240C, a magnetic sensor 240D, an acceleration sensor 240E, a grip sensor 240F, a proximity sensor 240G, a color sensor 240H (e.g., a red, green, blue (RGB) sensor). The sensor module 240 may also include an E-nose sensor, an electromyography (EMG) sensor, an electroencephalogram (EEG) sensor, an electrocardiogram (ECG) sensor, an infrared (IR) sensor, an iris sensor, and/or a fingerprint sensor. The sensor module 240 may further include a control circuit for controlling at least one sensor therein. The electronic device, as part of the processor 210 or individually, may further include a processor configured to control the sensor module 240 and control the sensor module 240 while the processor 210 is sleeping.

The input device 250 may include various input circuitry including at least one of a touch panel 252, a (digital) pen sensor 254, a key 256, and an ultrasonic input device 258. The touch panel 252 may use at least one of, for example, capacitive, resistive, infrared, and ultrasonic methods. Additionally, the touch panel 252 may further include a control circuit. The touch panel 252 may further include a tactile layer to provide a tactile response to a user. The (digital) pen sensor 254 may include, for example, part of a touch panel or a sheet for recognition. The key 256 may include, for example, a physical button, a touch key, an optical key, or a keypad. The ultrasonic input device 258 may detect ultrasonic waves from a microphone 288 and check data corresponding to the detected ultrasonic waves.

The display 260 (e.g., the display 160) may include at least one of a panel 262, a hologram device 264, a projector 266, and/or a control circuit for controlling them. The panel 262 may be flexible, transparent, or wearable, for example. The panel 262 and the touch panel 252 may be configured with one or more modules. The panel 262 may include a pressure sensor (or a force sensor) for measuring a pressure of the user touch. The pressure sensor may be integrated with the touch panel 252, or include one or more sensors separately from the touch panel 252. The hologram device 264 may show three-dimensional images in the air by using the interference of light. The projector 266 may display an image by projecting light on a screen. The screen, for example, may be placed inside or outside the electronic device 201.

The interface 270 includes various interface circuitry, such as an HDMI 272, a USB 274, an optical interface 276, or a D-subminiature (D-sub) 278. The interface 270 may be included in, for example, the communication interface 170 of FIG. 1. Additionally or alternately, the interface 270 may include a mobile high-definition link (MHL) interface, a SD card/MMC interface, or an infrared data association (IrDA) standard interface.

The audio module 280, for example, may convert sounds into electrical signals and convert electrical signals into sounds. At least some components of the audio module 280 may be included in, for example the input/output interface 150 of FIG. 1. The audio module 280 may process sound information input or output through a speaker 282, a receiver 284, an earphone 286, or the microphone 288. The camera module 291, as a device for capturing still images and videos, may include one or more image sensors (e.g., a front sensor or a rear sensor), a lens, an image signal processor (ISP), or a flash (e.g., an LED or a xenon lamp). The power management module 295, for example, may manage the power of the electronic device 201. According to an embodiment of the present disclosure, the power management module 295 may include a power management IC (PMIC), a charger IC, or a battery gauge, for example. The PMIC may have a wired and/or wireless charging method. The wireless charging method may include, for example, a magnetic resonance method, a magnetic induction method, or an electromagnetic method, and may further include an additional circuit for wireless charging, for example, a coil loop, a resonant circuit, or a rectifier circuit. The battery gauge may measure the remaining charge capacity of the battery 296, or a voltage, current, or temperature of the battery 296 during charging. The battery 296 may include, for example, a rechargeable battery and/or a solar battery.

The indicator 297 may display a specific state of the electronic device 201 or part thereof (e.g., the processor 210), for example, a booting state, a message state, or a charging state. The motor 298 may convert electrical signals into a mechanical vibration and generate a vibration or haptic effect. The electronic device 201 may include a mobile TV supporting device (e.g., a GPU) for processing media data according to standards such as digital multimedia broadcasting (DMB), digital video broadcasting (DVB), or MediaFlo™. Each of the above-described components of the electronic device may be configured with at least one component and the name of a corresponding component may vary according to the kind of electronic device. According to an embodiment of the present disclosure, the electronic device 201 may be configured to include at least one of the above-described components or an additional component, or to not include some of the above-described components. Additionally, some of components in an electronic device are configured as one entity, so that functions of previous corresponding components are performed identically.

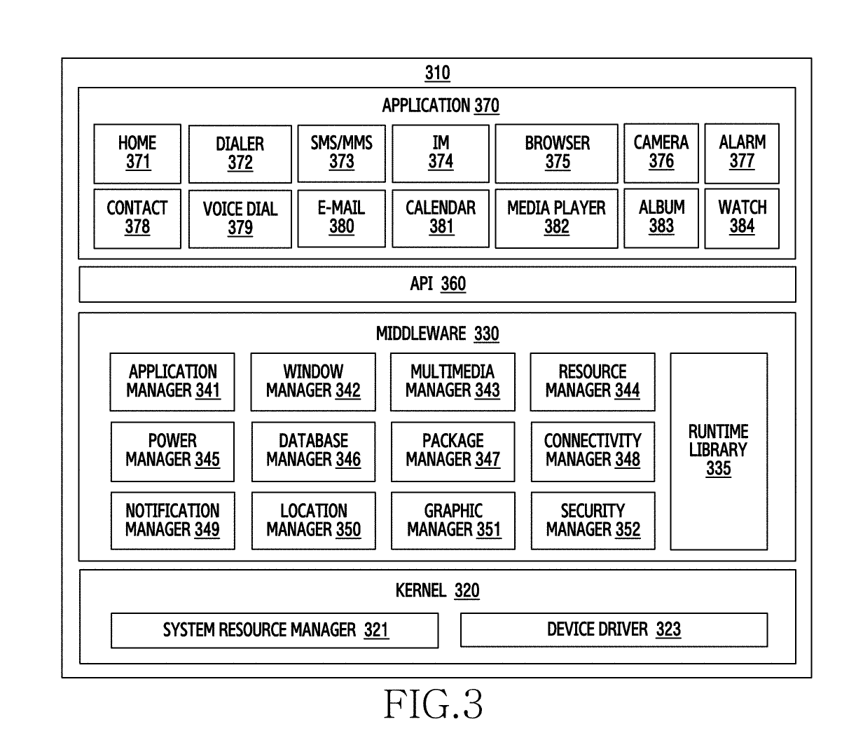

FIG. 3 is a block diagram of a program module, according to an embodiment of the present disclosure.

A program module 310 (e.g., the program 140) may include an OS for controlling a resource relating to the electronic device 101 and/or the applications 147 running on the OS. The OS may include, for example, Android™, iOS™, Windows™, Symbian™, Tizen™, or Bada™. Referring to FIG. 3, the program module 310 includes a kernel 320 (e.g., the kernel 141), a middleware 330 (e.g., the middleware 143), an API 360 (e.g., the API 145), and/or an application 370 (e.g., the applications 147). At least part of the program module 310 may be preloaded on an electronic device or may be downloaded from an external electronic device (e.g., the first external electronic device 102, the second external electronic device 104, or the server 106).

The kernel 320 includes, for example, at least one of a system resource manager 321 and/or a device driver 323. The system resource manager 321 may control, allocate, or retrieve a system resource. According to an embodiment of the present disclosure, the system resource manager 321 may include a process management unit, a memory management unit, or a file system management unit. The device driver 323 may include, for example, a display driver, a camera driver, a Bluetooth™ driver, a shared memory driver, a USB driver, a keypad driver, a Wi-Fi driver, an audio driver, or an inter-process communication (IPC) driver.

The middleware 330, for example, may provide a function commonly required by the application 370, or may provide various functions to the application 370 through the API 360 in order to allow the application 370 to efficiently use a limited system resource inside the electronic device. The middleware 330 includes at least one of a runtime library 335, an application manager 341, a window manager 342, a multimedia manager 343, a resource manager 344, a power manager 345, a database manager 346, a package manager 347, a connectivity manager 348, a notification manager 349, a location manager 350, a graphic manager 351, and a security manager 352.

The runtime library 335 may include, for example, a library module used by a compiler to add a new function through a programming language while the application 370 is running. The runtime library 335 may manage input/output, manage memory, or arithmetic function processing. The application manager 341, for example, may manage the life cycle of the applications 370. The window manager 342 may manage a GUI resource used in a screen. The multimedia manager 343 may recognize a format for playing various media files and encode or decode a media file by using the codec in a corresponding format. The resource manager 344 may manage a source code of the application 3740 or a memory space. The power manager 345 may manage the capacity, temperature, and/or power of the battery, and determine or provide power information for an operation of the electronic device using corresponding information among the capacity, temperature, and/or power of the battery. The power manager 345 may operate together with a basic input/output system (BIOS). The database manager 346 may create, search, or modify a database used in the application 370. The package manager 347 may manage installation or updating of an application distributed in a package file format.

The connectivity manager 348 may manage, for example, a wireless connection. The notification manager 349 may provide an event, such as incoming messages, appointments, and proximity alerts, to the user. The location manager 350 may manage location information of an electronic device. The graphic manager 351 may manage a graphic effect to be provided to the user or a user interface relating thereto. The security manager 352 may provide, for example, system security or user authentication. The middleware 330 may include a telephony manager for managing a voice or video call function of the electronic device, or a middleware module for combining various functions of the above-described components. The middleware 330 may provide a module specialized for each type of OS. The middleware 330 may dynamically delete part of the existing components or add new components. The API 360, as a set of API programming functions, may be provided as another configuration according to the OS. For example, Android or iOS may provide one API set for each platform, and Tizen may provide two or more API sets for each platform.

The application 370 includes at least one of a home 371, a dialer 372, an SMS/multimedia messaging system (MMS) 373, an instant message (IM) 374, a browser 375, a camera 376, an alarm 377, a contact 378, a voice dial 379, an e-mail 380, a calendar 381, a media player 382, an album 383, a clock 384. Additionally, the application 370 may include health care (e.g., measure an exercise amount or blood sugar level), or environmental information (e.g., air pressure, humidity, or temperature information) application. The application 370 may include an information exchange application for supporting information exchange between the electronic device and an external electronic device. The information exchange application may include, for example, a notification relay application for relaying specific information to the external device or a device management application for managing the external electronic device. The notification relay application may relay notification information from another application of the electronic device to an external electronic device, or receive and forward notification information from an external electronic device to the user. The device management application, for example, may install, delete, or update a function (e.g., turn-on/turn off of the external electronic device itself (or some components) or display brightness (or resolution) adjustment) of an external electronic device communicating with the electronic device, or an application operating in the external electronic device. The application 370 may include a specific application (e.g., a health care application of a mobile medical device) according to a property of the external electronic device. The application 370 may include an application received from an external electronic device. At least part of the program module 310 may be implemented (e.g., executed) with software, firmware, hardware (e.g., the processor 210), or a combination of at least two of them, and include a module, a program, a routine, a set of instructions, or a process for executing one or more functions.

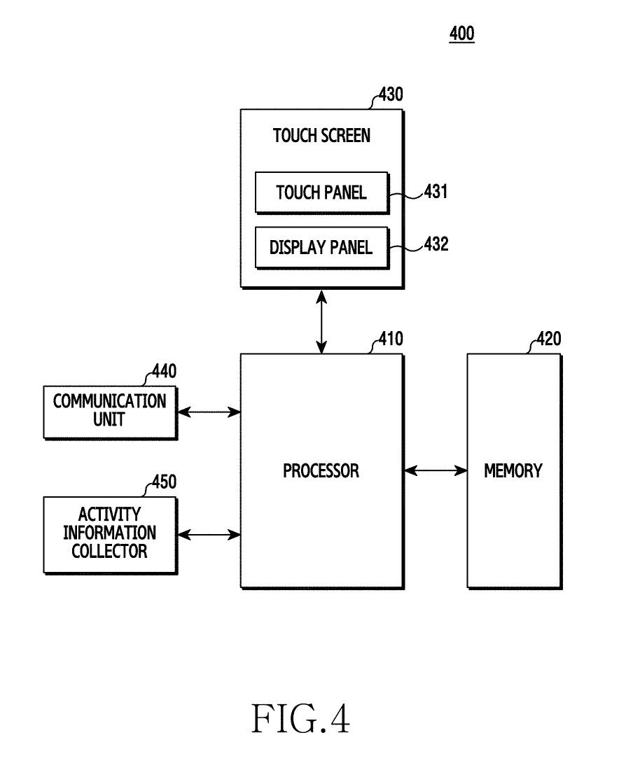

FIG. 4 is a block diagram of an electronic device according to an embodiment of the present disclosure.

Referring to FIG. 4, the electronic device 400 includes a processor 410, a memory 420, a touch screen 430, a communication unit 440, and an activity information collector 450. The electronic device 400 may include, for example, whole or part of the electronic device 101 of FIG. 1 or the electronic device 201 of FIG. 2.

The processor 410 (e.g., the processor 120, the processor 210) may control operations of the electronic device 400. The processor 410 may control the components of the electronic device 400. The processor 410 may receive commands and/or instructions from the memory 420, control the components according to the received commands and/or instructions, and execute various functions.

The processor 410 may include a CPU, an application processor (AP), a micro control unit (MCU), and the like. The processor 410 may include a single core processor or a multi-core processor. Alternatively, the processor 410 may be a multi-processor including a plurality of processors. The processor 410 may include an AP and a CP.

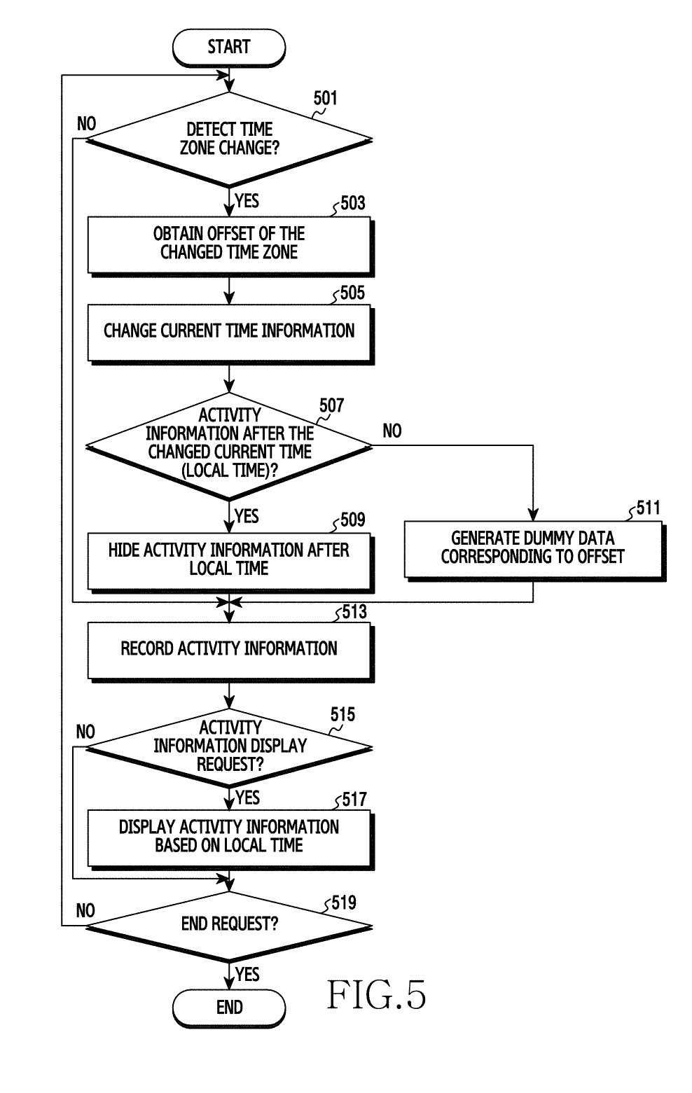

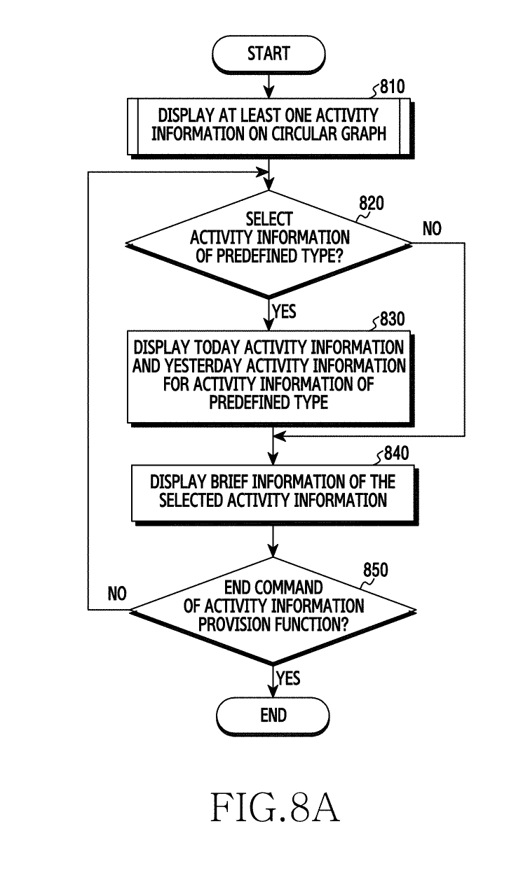

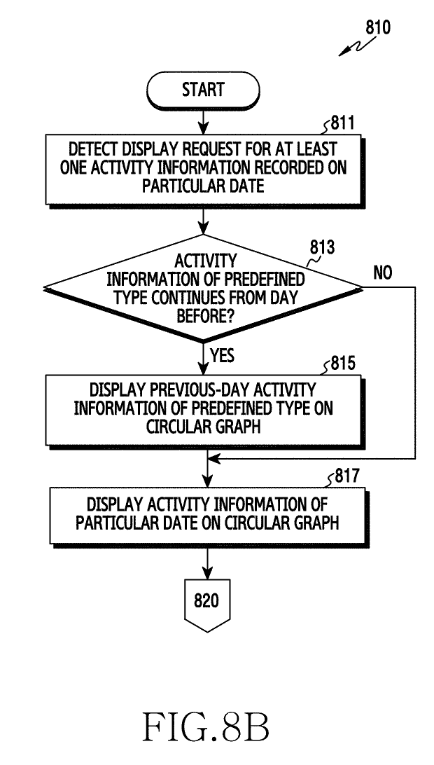

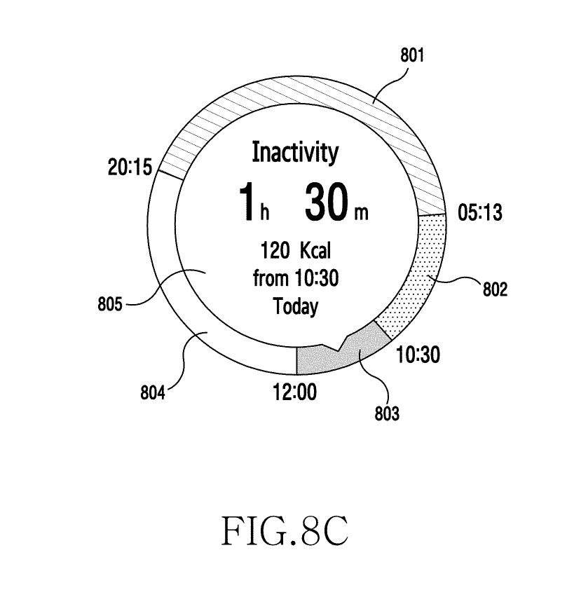

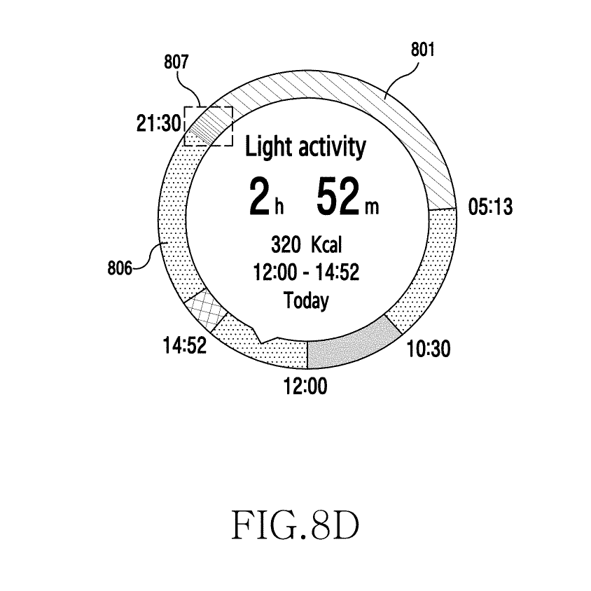

According to an embodiment of the present disclosure, the processor 410 may collect and provide activity information of the electronic device 400. In detail, the processor 410 may collect and record user's activity information through the activity information collector 450. The processor 410 may periodically collect and record the activity information. In storing the activity information, the processor 410 may also store a UTC and an offset. The offset refers to a difference in local time when moving from one time zone to another time zone. The processor 410 may provide the collected user activity information based on 24 hours using a circular, oval, or quadrangular graph, as described in FIG. 5, FIG. 8A, and FIG. 8B.

The memory 420 (e.g., the memory 130, the memory 230) may store various programs for operating the electronic device 400, and store data generated or downloaded in executions of the various programs. The memory 420 may store various commands and/or instructions for operating the processor 410. The memory 420 may include at least one of an internal memory or an external memory.

According to an embodiment of the present disclosure, the memory 420 may store at least one activity information collected through the activity information collector 450. The activity information may be stored in a database. The memory 420 may store the activity information as shown in Table 1.

TABLE 1

| Activity Information | UTC | Offset | Local Time |
|---|---|---|---|
| light activity | 01:00 | 0 | 01:00 |
| exercising | 02:00 | 0 | 02:00 |
| not wearing | 03:00 | −1 | 02:00 |
| inactivity | 04:00 | −1 | 03:00 |
| sleep | 05:00 | −1 | 04:00 |
| light activity | 06:00 | −1 | 05:00 |
| inactivity | 07:00 | −1 | 06:00 |
| exercising | 08:00 | 0 | 08:00 |
| inactivity | 09:00 | 0 | 09:00 |
| light activity | 10:00 | 0 | 10:00 |

Referring to Table 1, each activity information may be mapped to the UTC and the offset. In Table 1, the electronic device 400 moves at 03:00 based on the UTC into a time zone where the offset decreases and moves at 08:00 into a time zone where the offset increases. In the time zone where the offset decreases, the activity information labeled “exercising” at UTC 02:00 may be overwritten by the activity information labeled “not wearing” information of UTC 03:00. When the offset increases, dummy data may be generated between “inactivity” information at UTC 07:00 and “exercising” information at UTC 08:00. When displaying the activity information on a circular graph, the electronic device 400 may blank a corresponding portion between 07:00 and 08:00.

The touch screen 430 (e.g., the display 160, the display 260) may provide an input function and an output function. The touch screen 430 may include a touch panel 431 and a display panel 432. The touch screen 430 may be formed in a circular, oval, or quadrangular shape.

The touch panel 431 may provide the input function. The touch panel 431 may detect (or recognize) a change of a physical characteristic (e.g., capacitance, frequency, etc.) according to various touch inputs (e.g., tap, double tap, touch, swipe, multi-touch, press, etc.) using an input means such as a finger, a stylus, or a digital pen, and send the change to the processor 410.

The display panel 432 may provide the output function. The display panel 432 may use, for example, an LCD, an LED display, an OLED display, a MEMS display, or an electronic paper display. The display panel 432 may be flexible, transparent, or wearable.

The display panel 432 may display, for example, various content (e.g., text, image, video, icon, and/or symbol). The display panel 432 may display the collected activity information using a graph. The display panel 432 may display the activity information using a circular, oval, or quadrangular graph.

The communication unit 440 (e.g., the communication interface 170, the communication module 220) may perform a communication function. The communication unit 440 may conduct wired or wireless communication. The communication unit 440 may include a USB communication module, a Wi-Fi communication module, a BT communication module, an NFC module, and a GPS module. According to an embodiment of the present disclosure, at least some (e.g., two or more) of the Wi-Fi communication module, the BT communication module, the NFC module, and the GPS module may be included in one IC or an IC package.

According to an embodiment of the present disclosure, the communication unit 440 may receive date and time information from another electronic device (a parent terminal) connected using short-range wireless communication, or receive time (or local) information from a base station in the network 162.

The activity information collector 450 may collect the user's activity information. The activity information collector 450 may periodically collect the activity information. The activity information collector 450 may include various sensors. The activity information collector 450 may include an acceleration sensor, a geomagnetic sensor, a gravity sensor, an illuminance sensor, a location information sensor (e.g., GPS), a biometric sensor (e.g., heart rate sensor), and a pressure sensor.

The electronic device 400 may not include some of the abovementioned components. Alternatively, the electronic device 400 may further include at least one other component (e.g., an input module (e.g., a rotary bezel, a power key, etc.), an audio processing module, a camera module, a digital broadcasting module, etc.) equivalent to the above-stated components.

According to an embodiment of the present disclosure, an electronic device includes at least one sensor, a memory, a display, and at least one processor functionally coupled with the at least one sensor, the memory, and the display, wherein the at least one processor is configured to control the at least one sensor to collect activity information about a user's activity, control the memory to store the collected at least one activity information, and control the display to display at least one activity information collected on a particular date, using a 24-hour graph in response to an activity information display request, and control the display to display, when activity information continues from a previous day of the particular date to the particular date and is of a particular type, previous-day activity information of the particular type on the 24-hour graph.

According to an embodiment of the present disclosure, when activity information conducted on the particular date overlaps the previous-day activity information of the activity information of the particular type, the processor displays the activity information of the particular date on the previous-day activity information.

According to an embodiment of the present disclosure, the processor differently displays an overlapping part of the activity information of the particular date and the previous-day activity information of the particular type.

According to an embodiment of the present disclosure, when at least part of the previous day activity information of the particular type is overlapped and hidden and the activity information of the particular type is selected, the processor displays the entire activity information of the particular type on the 24-hour graph.

According to an embodiment of the present disclosure, the activity information of the particular type may be sleep information.

According to an embodiment of the present disclosure, the processor differently displays activity information, which is not of the particular type, continuing from the previous day to the particular date, as separate activity information.

According to an embodiment of the present disclosure, when an input on the separate activity information is detected, the processor displays all of the separate activity information on the 24-hour graph by further displaying previous-day activity information of the separate activity information on the 24-hour graph.

According to an embodiment of the present disclosure, the processor stores the at least one activity information by mapping the at least one activity information to a UTC and a time offset.

According to an embodiment of the present disclosure, when detecting a time zone change and activity information after a current time of the changed time zone, the processor hides the activity information.

FIG. 5 is a flowchart of a method for storing and providing user activity information in an electronic device, according to an embodiment of the present disclosure.

Referring to FIG. 5, the electronic device (e.g., the processor 120 of the electronic device 101, the processor 210 of the electronic device 201, or the processor 410 of the electronic device 400) detects a time zone change in operation 501. When supporting a mobile communication function, the electronic device may detect the time zone change based on IMSI information received from a base station. When a mobile country code of the IMSI information changes, the electronic device may recognize the time zone change. When not supporting the mobile communication function, the electronic device may receive time zone information from a parent terminal (e.g., a smart phone) connected using the short-range wireless communication. The electronic device may receive daylight savings time information together with the time zone information.

When the time zone does not change in operation 501, the electronic device records (stores) activity information collected through at least one sensor in operation 513.

When the time zone changes in operation 501, the electronic device obtains an offset of the changed time zone in operation 503. The electronic device may recognize a current location (country) based on the mobile country code of the IMSI information, and obtain the offset of the changed time zone. The electronic device may receive offset information from the parent terminal.

In a region which observes daylight savings time, the electronic device may obtain the offset by observing the daylight savings time.

In operation 505, the electronic device changes current time information with time information of the changed time zone (hereafter, local time). The electronic device may change the current time information by adding the offset to the UTC.

In operation 507, the electronic device determines whether a database recording the activity information includes activity information after the local time.

When detecting the activity information after the local time in operation 507, the electronic device hides the activity information after the local time in operation 509. When detecting no activity information after the local time in operation 507, the electronic device generates dummy data (empty area) corresponding to the offset in operation 511.

In operation 513, the electronic device records (stores) user activity information collected through at least one sensor. The electronic device may collect the activity information on a periodic basis and store it with the UTC and the offset.

In operation 515, the electronic device determines whether the activity information display is requested. When receiving no display request of the activity information, the electronic device goes to operation 519. When receiving the display request of the activity information, the electronic device displays the recorded activity information based on the local time in operation 517. The electronic device may display the activity information using a circular graph.

In operation 519, the electronic device determines whether an end request is detected. The end request may request to stop recording the user activity information.

When not detecting the end request, the electronic device returns to operation 501. Upon detecting the end request, the electronic device stops recording the user activity information.

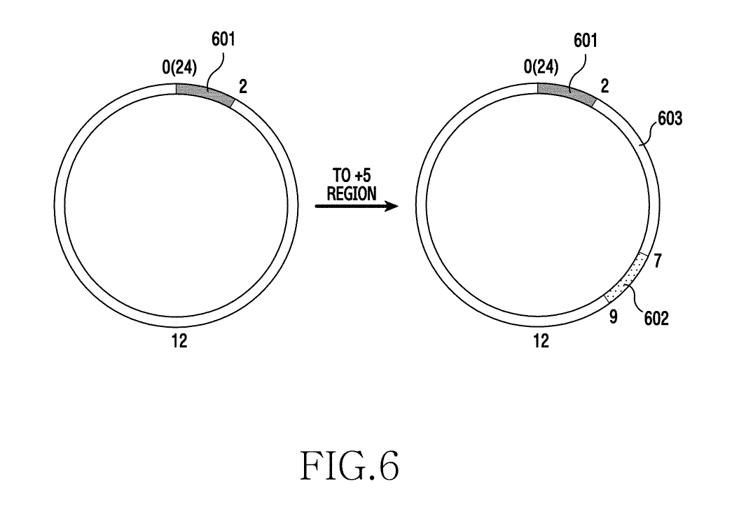

FIG. 6 is a diagram of activity information displayed when moving into a region where a time offset increases, according to an embodiment of the present disclosure.

Referring to FIG. 6, assuming that the electronic device collects first activity information from 00:00 to 02:00 in a local time region, changes to a time zone of an offset “+5” (plus 5 hours) at 02:00 (e.g., automatically changes based on the regional movement or manually changes according to the user), and continuously collects second activity information for two hours, the electronic device may display the first activity information 601 from 00:00 to 02:00 on a circular graph and display the second activity information 602 from 07:00 to 09:00 which are the local time (UTC+offset) of the changed region. The circular graph may display no activity information 603 corresponding to the offset. The electronic device may visually differentiate the first activity information 601 from the second activity information 602 to indicate that they are collected in different time zones. When the first activity information 601 and the second activity information 602 are of the same type, the electronic device may differentiate the displaying of the second activity information 602 from the first activity information 601. Alternatively, the electronic device may display time zone information (e.g., text, number, symbol, icon (e.g., a national flag of a corresponding region)) near the graphs of the first activity information 601 and the second activity information 602.

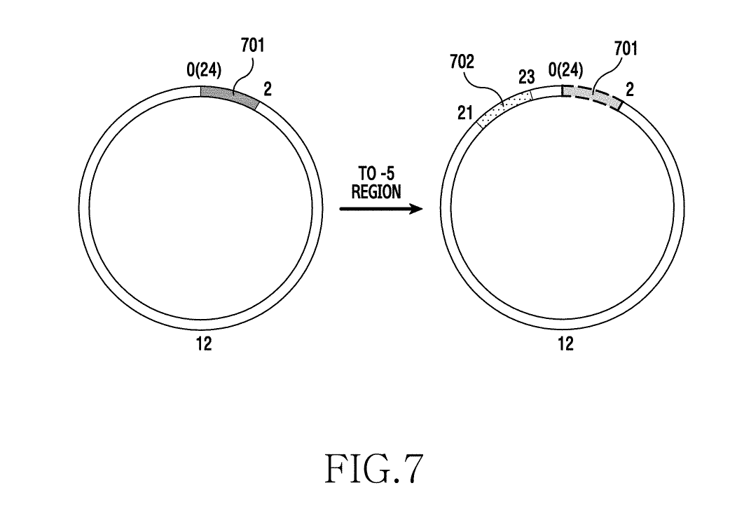

FIG. 7 is a diagram of activity information displayed when moving into a region where a time offset decreases according to an embodiment of the present disclosure.

Referring to FIG. 7, assuming that the electronic device collects first activity information 701 from 00:00 to 02:00 in a UTC region, changes to a time zone of an offset “−5” (minus 5 hours) at 02:00 (e.g., automatically changes based on the regional movement or manually changes according to the user), and continuously collects second activity information for two hours, the electronic device may display the second activity information 702 from 21:00 to 23:00 in a circular graph. In so doing, the electronic device may hide the first activity information 701 from 00:00 to 02:00 because it is future data based on a current time. The first activity information 701 is hidden and is not actually deleted. When the user moves back to the UTC region, the first activity information 701 may be displayed again on the circular graph. According to an embodiment of the present disclosure, when the time passes 00:00 with the offset “−5”, the first activity information 701 may be overwritten by new activity information.

To indicate that the second activity information 702 is collected in the changed time zone, the electronic device may differentiate the displaying of the second activity information 702. The electronic device may differentiate color and transparency of the second activity information 702 from the first activity information 701. Alternatively, the electronic device may display time zone information (e.g., text, number, symbol, icon (e.g., a national flag of a corresponding region)) near the graph of the second activity information 702.

FIG. 8A is a flowchart of a method for providing user activity information of an electronic device, according to an embodiment of the present disclosure.

FIG. 8B is a flowchart of a method for displaying user activity information of an electronic device using a circular graph, according to an embodiment of the present disclosure.

FIGS. 8C and 8D are diagrams of a circular graph displaying user activity information of an electronic device, according to an embodiment of the present disclosure.

Referring to FIGS. 8A through 8D, in operation 810, the electronic device (e.g., the processor 120 of the electronic device 101, the processor 210 of the electronic device 201, or the processor 410 of the electronic device 400) may display at least one activity information on a circular graph. The circular graph may visually display various user activity information on a particular date based on 24 hours.

In FIG. 8B, the electronic device displays at least one activity information on the circular graph, the electronic device may detect a display request for at least one activity information recorded on a particular date (e.g., today) in operation 811.

In operation 813, the electronic device determines whether activity information of a predefined type continues from the day before (e.g., yesterday). The activity information of the predefined type may include sleep activity information.

When detecting no activity information of the predefined type in operation 813, the electronic device proceeds to operation 817.

By contrast, when detecting the activity information of the predefined type in operation 813, the electronic device displays yesterday's activity information of the predefined type on the circular graph of the particular date in operation 815.

In operation 817, the electronic device displays activity information of a particular date on the circular graph. In so doing, when the activity information of the particular date overlaps yesterday's activity information of the predefined type, the electronic device displays the activity information of the particular date above yesterday's activity information of the predefined type. Assuming that the user sleeps from yesterday 20:15 to today 05:13, gets up and performs light activity until 10:30, and is inactive until 12:00, the electronic device displays a circular graph as shown in FIG. 8C. In FIG. 8C, reference numeral 801 indicates sleep information, reference numeral 802 indicates light activity information, reference numeral 803 indicates inactivity information, and reference numeral 804 indicates no data after a current time. The electronic device may display brief information 805 about the inactivity information 803 currently selected, inside the circular graph. As such, for the sleep information 801, the electronic device may display yesterday's information and today's information together because total sleep time is generally more meaningful to the user.

Referring to FIG. 8D, when time passes and the start time of the sleep information exceeds 20:15, new activity information 806 and the sleep information 801 may overlap. In this case, the electronic device may display the new activity information 806 above the sleep information 801. To indicate that the two activity information overlap, the electronic device may provide a graphical effect or visual indicator. The electronic device may apply a gradation effect 807 to the overlapping area of the two activity information. According to an embodiment of the present disclosure, the electronic device may adjust transparency of the upper activity information to show the lower activity (sleep) information. Alternatively, the electronic device may display a preset symbol (a sign, a letter, an icon, etc.) in the overlapping part of the two activity information.

Referring to FIG. 8A, in operation 820, the electronic device determines whether activity information of a predefined type is selected from activity information displayed on the circular graph. The activity information of the predefined type may be selected in various input methods. The activity information of the predefined type may be selected using a rotary bezel surrounding a circular display. Alternatively, the activity information of the predefined type may be selected by using touch input.

When the activity information of the predefined type is not selected in operation 820, the electronic device goes to operation 840. When the activity information of the predefined type is selected in operation 820, the electronic device displays today's activity information and yesterday's activity information of the activity information of the predefined type in operation 830. That is, the electronic device may display the entire activity information of the predefined type on the circular graph. The electronic device may display yesterday's sleep information which is not displayed on the circular graph of the particulate date, on the circular graph of the particular date, and display the entire sleep information on the circular graph. According to an embodiment of the present disclosure, when displaying yesterday's sleep information, the electronic device may provide an animation effect which expands the graph.

In operation 840, the electronic device displays brief information about the selected activity information inside the circular graph. When the inactivity information is selected, the electronic device may display a title, an inactivity time, calories burned, a start time, and a date inside the graph as shown in FIG. 8C. In FIG. 8C, the start time information “from 10:30” means that the inactivity begins at 10:30 to the present time and does not end. When the light activity information is selected, the electronic device may display a title, an activity time, calories burned, a start time, an end time, and a date inside the graph as shown in FIG. 8D.

In operation 850, the electronic device determines whether an end command of the activity information function is input. When the end command is not input, the electronic device returns to operation 820 and repeats the above operations. When the end command is input, the electronic device ends the activity information function.

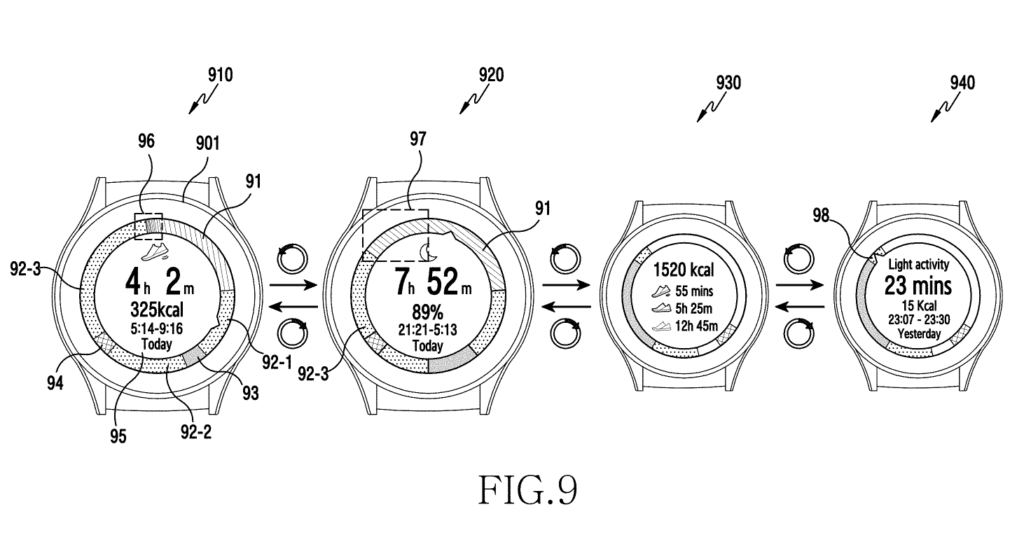

FIG. 9 is a diagram of displaying user activity information on an electronic device, according to an embodiment of the present disclosure.

Referring to FIG. 9, the electronic device may display activity information recorded on a particular date on a circular graph according to a user's request. The circular graph 910 includes sleep 91, a first light activity 92-1, inactivity 93, a second light activity 92-2, exercising 94, and a third light activity 92-3. In the circular graph 910, the first light activity 92-1 is selected. Brief information 95 about the selected first light activity 92-1 may be displayed inside the circular graph. The brief information 95 includes an activity time, calories burned, start and end times, a date, and the like. When the user conducts the third light activity 92-3 after yesterday's sleep start time, the third light activity 92-3 may partly overlap the sleep information 91. In this case, the electronic device may apply a graphical effect 96 (e.g., gradation) to the sleep information 91 and notify the user of the hidden information. The graphical effect may move according to the third light activity 92-3 of the user. When the user starts to sleep yesterday at 22:00 and starts the light activity today from 22:00, the electronic device may apply the graphical effect to the left end of the sleep information 91 from 22:00 and move the graphical effect to the right as the time passes. At 22:30, the graphical effect is placed near 22:30.

When a rotary bezel 901 of the electronic device rotates left in the graph 910, the sleep information 91 on the left side of the first light activity 92-1 may be selected on a circular graph 920. When the sleep information 91 is selected, the electronic device may display part 97 of the sleep information 91 hidden by the third light activity 92-3 and display the entire sleep time on the circular graph.

The electronic device may display yesterday's activity information on the graph of today's activity information in relation to the activity information (e.g., sleep) of a particular type. Hence, the user may easily obtain his/her sleep time on the graph. The brief information about the selected sleep information 91 may be displayed inside the circular graph. The brief information may include a total sleep time, a deep sleep rate, sleep start/end times, and a date.

When the rotary bezel 901 of the electronic device rotates left on the circular graph 920 (with the initial activity information selected from the displayed activity information), the electronic device may display brief information about yesterday's user activity information on a circular graph 930.

When the rotary bezel 901 of the electronic device rotates left on the circular graph 930, light activity information 98 which is the last activity information of yesterday may be selected on a circular graph 940.

When the rotary bezel 901 of the electronic device rotates right on the circular graph 940, the electronic device may switch to the graph 930. When the rotary bezel 901 of the electronic device rotates right on the circular graph 930, the electronic device may switch to the graph 920. When the rotary bezel 901 of the electronic device rotates right on the circular graph 920, the electronic device may switch to the graph 910.

While the activity information is selected using the rotary bezel 910 in FIG. 9, the present disclosure is not limited to such and the activity information may be selected using various inputs (e.g., touch and/or pressure, gesture, input using the stem of an electronic device of a watch type, etc.).

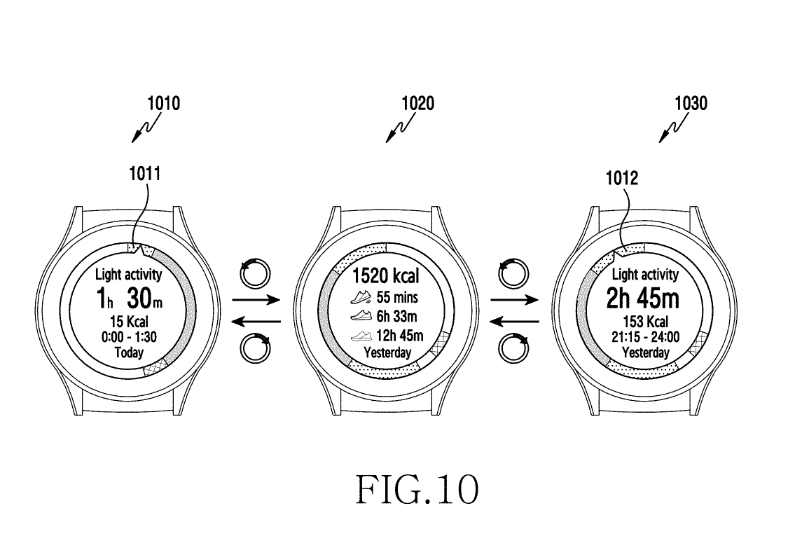

FIG. 10 is a diagram of user activity information provided from an electronic device, according to an embodiment of the present disclosure.

Referring to FIG. 10, when activity information which is not of a predefined type continues from the day before, the electronic device may distinguish it based on a date. When the user continues a light activity from yesterday 21:15 to today 01:30, the electronic device may display the light activity 1011 from 00:00 to 01:30 today on a circular graph 1010 showing today's activity information. At this time, brief information may be displayed related to the light activity 1011 conducted from 00:00 to 01:30 today.

When a rotary bezel of the electronic device rotates left on the circular graph 1010 (with the initial activity information selected from today's activity information), the electronic device may display brief information about the entire user activity information of yesterday on a circular graph 1020. The electronic device may display a total calories burned during the day before, a total light activity time, a total exercising time, a total walk time, and the like.

When the rotary bezel of the electronic device rotates left on the circular graph 1020, light activity information 1012 which is yesterday's last activity information may be selected on a circular graph 1030. As such, even when the activity information continues from the day before to the present, the electronic device may distinguish the activity information based on the date because the activity during the day is meaningful to the user.

The continuous activity information may be divided based on the date and displayed as, but not limited to, separate information. When a predefined input (e.g., long touch, press, etc.) occurs on the continuous activity information, the electronic device may display the continuous activity information performed from the day before, such as sleep information, as one information.

According to an embodiment of the present disclosure, a method for providing activity information in an electronic device includes collecting and storing activity information of a user's activity, and displaying activity information collected on a particular date, using a 24-hour graph in response to an activity information display request, wherein displaying the activity information using the graph includes, when activity information continues from a previous day of the particular date to the particular date and is of a particular type, displaying previous-day activity information of the particular type on the 24-hour graph.

According to an embodiment of the present disclosure, the method further includes, when activity information conducted on the particular date overlaps the previous-day activity information of the particular type, displaying the activity information of the particular date on the previous-day activity information of the particular type.

According to an embodiment of the present disclosure, the method further includes differently displaying an overlapping part of the activity information of the particular date and the previous-day activity information of the particular type.

According to an embodiment of the present disclosure, the method further includes, when at least part of the previous-day activity information of the particular type is hidden and the activity information of the particular type is selected, displaying entire activity information of the particular type on the graph by displaying the previous-day activity information of the particular type on top of a display.

According to an embodiment of the present disclosure, the activity information of the particular type is sleep information.

According to an embodiment of the present disclosure, the further includes differently displaying the 24-hour graph activity information which continues from the previous day to the particular date but is not of the particular type, as separate activity information.

According to an embodiment of the present disclosure, the method further includes, when an input on the separate activity information is detected, displaying all of the separate activity information on the graph by further displaying previous-day activity information of the separate activity information on the 24-hour graph.

According to an embodiment of the present disclosure, collecting and storing the activity information includes storing the activity information by mapping the activity information to a universal time coordinated (UTC) and a time offset.

According to an embodiment of the present disclosure, the storing further includes, when detecting a time zone change and activity information after a current time of the changed time zone, hiding the activity information.

When the time zone changes, the user's activity information may be provided based on the time information (the local time) of the changed time zone (e.g., the current user region).

Yesterday's activity information may be displayed on today's graph. The user may easily view the entire activity information (yesterday's activity information plus today's activity information) of the particular type selected on today's graph.

The term “module”, as used herein, may refer, for example, to a unit including hardware, software, and firmware, or any suitable combination thereof. The term “module” may be interchangeably used with terms such as “unit”, “logic”, “logical block”, “component”, “circuit”, and the like. A module may be a minimum unit of an integral component or may be a part thereof. A module may be a minimum unit for performing one or more functions or may be a part thereof. A module may be mechanically or electrically implemented. A module, according to an embodiment of the present disclosure, may include, at least one of a dedicated processor, a CPU, an application-specific integrated circuit (ASIC) chip, a field-programmable gate arrays (FPGAs), and a programmable-logic device, which are known or will be developed and which perform certain operations.

At least some parts of a device (e.g., modules or functions thereof) or a method (e.g., operations), based on embodiments of the present disclosure, may be implemented with an instruction stored in a non-transitory computer-readable storage medium (e.g., the memory 130) as a program module. When the instruction is executed by a processor (e.g., the processor 120), the processor may perform a function corresponding to the instruction. The non-transitory computer readable recording medium may include, for example, a hard disk, a floppy disc, a magnetic medium (e.g., a magnetic tape), an optical storage medium (e.g., a compact disc-ROM (CD-ROM) or a DVD, a magnetic-optic medium (e.g., a floptical disc)), and an internal memory. The instruction may include code created by a compiler or code executable by an interpreter.

According to an embodiment of the present disclosure, a computer-readable recording medium may store at least one instruction. The at least one instruction may be set for at least one processor to perform at least one operation when the at least one instruction is executed by the at least one processor. The at least one operation may include collecting and storing activity information of a user's activity and displaying at least one activity information collected on a particular date, using a 24-hour graph in response to an activity information display request, and when activity information of a particular type continues from a previous day of the particular date to the particular date, displaying previous-day activity information of the particular type together on the graph.

The module or program module may further include at least one or more components among the aforementioned components, or may omit some of them, or may further include additional other components. Operations performed by a module, program module, or other components of certain embodiments of the present disclosure may be executed in a sequential, parallel, repetitive, or heuristic manner. In addition, some of the operations may be executed in a different order or may be omitted, or other operations may be added.

The embodiments of the present disclosure disclosed herein and illustrated in the drawings are merely examples presented in order to describe technical details of the present disclosure and to aid in the understanding of the present disclosure, and are not intended to limit the scope of the present disclosure. Therefore, it should be understood that, in addition to the embodiments disclosed herein, all modifications and changes or modified and changed forms derived from the technical idea of the present disclosure fall within the scope of the present disclosure as defined by the appended claims and their equivalents.

What is claimed is:

1. An electronic device comprising:
at least one sensor;
a memory;
a display; and
at least one processor functionally coupled with the at least one sensor, the memory, and the display,
wherein the at least one processor is configured to:
control the at least one sensor to collect activity information about a user's activity,
control the memory to store the collected activity information,
control the display to display activity information collected on a particular date, using a 24-hour graph, in response to an activity information display request, and
control the display to display, when the activity information continues from a previous day of the particular date to the particular date and is of a particular type, previous-day activity information of the particular type on the 24-hour graph.

2. The electronic device of claim 1, wherein, when activity information conducted on the particular date overlaps the previous-day activity information of the particular type, the processor is further configured to display the activity information of the particular date as an overlap on the previous-day activity information.

3. The electronic device of claim 2, wherein the processor is further configured to differently display an overlapping part of the activity information of the particular date and the previous-day activity information of the particular type.

4. The electronic device of claim 2, wherein, when at least part of the previous-day activity information of the particular type is overlapped and hidden and the activity information of the particular type is selected, the processor is further configured to display entire activity information of the particular type on the 24-hour graph.

5. The electronic device of claim 1, wherein the activity information of the particular type is sleep information.

6. The electronic device of claim 1, wherein the processor is further configured to differently display activity information, which is not of the particular type, of activity information continuing from the previous day to the particular date, as separate activity information.

7. The electronic device of claim 6, wherein, when an input on the separate activity information is detected, the processor is further configured to display all of the separate activity information on the 24-hour graph by further displaying previous-day activity information of the separate activity information on the 24-hour graph.

8. The electronic device of claim 1, wherein the processor is further configured to store the activity information by mapping the activity information to a universal time coordinated (UTC) and a time offset.

9. The electronic device of claim 8, wherein, when a time zone change and activity information after a current time of the changed time zone is detected, the processor is further configured to hide the activity information.

10. A method for providing activity information in an electronic device, comprising:
collecting and storing activity information of a user's activity; and
displaying the activity information collected on a particular date, using a 24-hour graph in response to an activity information display request,
wherein displaying the activity information using the 24-hour graph comprises,
when the activity information continues from a previous day of the particular date to the particular date and is of a particular type, displaying previous-day activity information of the particular type on the 24-hour graph.

11. The method of claim 10, further comprising:
when activity information conducted on the particular date overlaps the previous-day activity information of the particular type, displaying the activity information of the particular date as an overlap on the previous-day activity information of the particular type.

12. The method of claim 11, further comprising:
differently displaying an overlapping part of the activity information of the particular date and the previous-day activity information of the particular type.

13. The method of claim 11, further comprising:
when at least part of the previous-day activity information of the particular type is hidden and the activity information of the particular type is selected, displaying entire activity information of the particular type on the 24-hour graph by displaying the previous-day activity information of the particular type on top of the overlapped display.

14. The method of claim 10, wherein the activity information of the particular type is sleep information.

15. The method of claim 11, wherein displaying the activity information using the 24-hour graph comprises:
differently displaying the 24-hour graph of the activity information which continues from the previous day to the particular date but is not of the particular type, as separate activity information.

16. The method of claim 15, further comprising:
when an input on the separate activity information is detected, displaying all of the separate activity information on the 24-hour graph by further displaying previous-day activity information of the separate activity information on the 24-hour graph.

17. The method of claim 10, wherein collecting and storing the activity information comprises:
storing the activity information by mapping the activity information to a universal time coordinated (UTC) and a time offset.

18. The method of claim 17, wherein storing further comprises:
when a time zone change and activity information after a current time of the changed time zone is detected, hiding the activity information.

19. A computer-readable recording medium for storing at least one instruction, the at least one instruction, when executed by at least one processor, causes the at least one processor to:
collect and store activity information of a user's activity; and
display activity information collected on a particular date, using a 24-hour graph, in response to an activity information display request, and when activity information of a particular type continues from a previous day of the particular date to the particular date, displaying previous-day activity information of the particular type on the 24-hourgraph.

20. The computer-readable recording medium of claim 19, wherein, when a time zone change and activity information after a current time of the changed time zone is detected, hiding the activity information.
